(19) United States
(12) Patent Application Publication
(10) Pub. No.: US 2005/0021087 A1
(43) Pub. Date: Jan. 27, 2005

(54) Sternum suture wire with teardrop ring

(76) Inventor: Tomoaki Koseki, Chiyoda-ku (JP)

Correspondence Address: Tomoaki Koseki, 17-2, Sotokanda 2-chome, Chiyoda-ku, Tokyo, 101-0021, JP

(21) Appl. No.: 10/861,852

(22) Filed: Jun. 4, 2004

(30) Foreign Application Priority Data
Jul. 23, 2003 (JP) 2003-278659

(51) Int. Cl. A61L 17/00

(52) U.S. Cl. 606/228

(57) ABSTRACT

A sternum suture wire with teardrop ring, wherein the center part of the wire is thick, both ends are made thin, and the two tip ends of the wire are shaped in teardrop rings. This invention provides a sternum suture wire which has least invasiveness onto the sternum, that can be passed through a sternum easily and speedily, and the wires do not tangle with each other inside the container case.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC or REFERENCE TO A “MICROFICHE APPENDIX”

Not Applicable

### BACKGROUND OF THE INVENTION

(1) Field of the Invention

This invention relates to a wire for sternum closure material used during a cardiac surgery.

(2) Description of Related Art including information disclosed under 37 CFR 1.97 and 1.98.

Material used for sternum suture can be categorized in two, one being sternum piercing needles attached to one end of a wire, and the other being wires with both ends to thin down, and the wire tip end is passed through a tip of a gimlet and pulled up after pierced through the sternum. The processing method making the two ends of the wire to thin down is already proposed in patent documents 1 and 2 and such. In patent document 3, there is a proposal of a surgical suture thread having a ring with the objective of facilitating the knot making at the time of knot fastening, and a method to conduct suture by using the ring part and pulling it up after piercing the sternum with a gimlet, in FIG. 3. Patent documents 4 and 5 propose a combination of the fore mentioned inventions, proposing a needle to be attached to an end of a wire that is thinned.

Patent Document 1

Patent publication number Heisei 10-33553

Patent Document 2

Patent publication nubmer 2002-78712 (P2002-78712A)

Patent Document 3

Patent publication number 2001-198131 (P2001-198131A)

Patent Document 4

Patent publication number 2002-224122 (P2002-224122A)

Patent Document 5

Patent publication number 2003-79632 (P2003-79632A)

When using the type wherein a needle is attached to one end of a wire has strong invasiveness onto the sternum, causing hemorrhage, and therefore time and labor is required for hemostasis. In cases of surgeries on aged patients, bone fractures could occur due to bone fragility.

When using the type with gimlet to pierce the sternum, when a hole is made at the tip end of the gimlet, passing a wire through the tiny hole is difficult and the procedure takes time and labor.

When using a thread with a ring, as stated in document 3, when using a soft thread material, the procedure of placing the ring on the tip end of the gimlet that is pierced to the other side of the sternum is troublesome, and as this requires supporting of the ring part with a finger, there is a risk of harming the hand with the gimlet tip. In a case of a metal wire, the ring part can be made into a relatively large ring in advance and approach from the foreside, but when the form is made in a true circle, a multiple number of wires tangle inside the container case, and it is difficult to take the wires out.

### BRIEF SUMMARY OF THE INVENTION

To solve the above issues, this invention proposes a sternum suture wire with teardrop shape ring, wherein the center part of the wire is thick, the two ends being thin, and the two tip ends of the wire being formed in teardrop shape rings.

As explained above, with this invention, there is no invasiveness onto the sternum as when utilizing a wire with needle attached, and this invention enables piercing the wire through the sternum with easy operation of just placing the ring on the hook of the gimlet, shortening the surgery time. Taking out the wire from its container is easy, without any tangling, and a wire can be pulled out smoothly.

### BRIEF DESCRIPTION OF THE SEVERAL VIEWS OF THE DRAWINGS

FIG. 1 is an explanatory drawing of a wire with teardrop shape ring, being taken out of a container.

FIG. 2 is an explanatory drawing of a wire with teardrop shape ring, during usage.

### DETAILED DESCRIPTION OF THE INVENTION

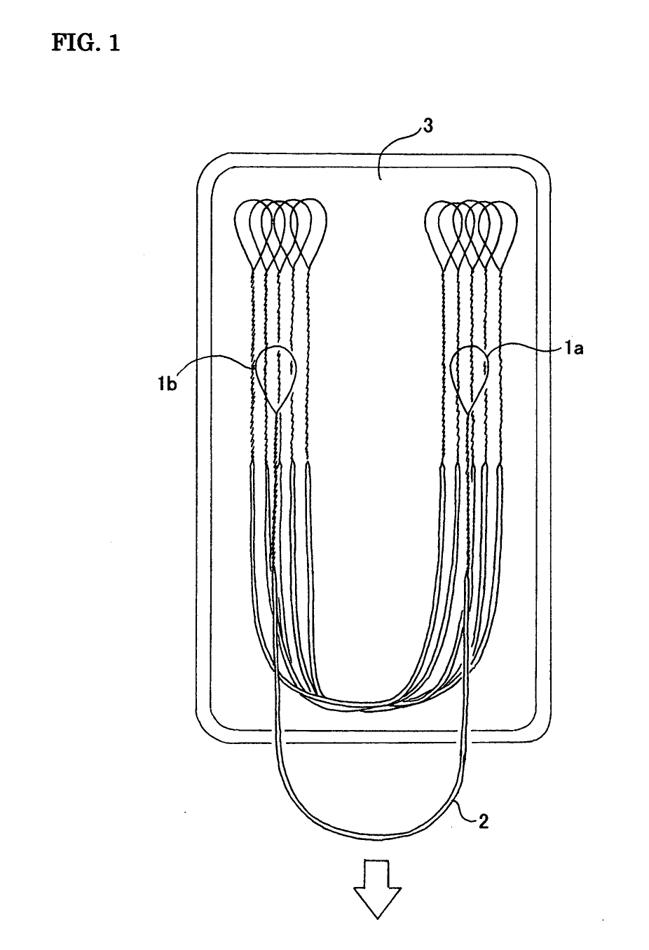

Hereafter, a description of the preferred embodiments of this invention is made in reference to drawings. FIG. 1 shows one embodiment of this invention, showing how a wire is taken out of its container. The material of the wire is SUS 316L, the center thick part of the wire has the diameter of 0.8 mm˜0.9 mm, the length being approximately 30 cm, and on the two ends of the wire, another wire is attached, made of material SUS316L, its outer diameter being 0.45 mm, the length being approximately 20 cm, twisted with the manufacturing method according to claim 2, the shape of the tip end being in teardrop shape ring 1a, 1b. In a cardiac surgery, five or six wires are usually used, and often five or six wires are set inside a container case 3. A typical procedure would be to first pull out the thick part of the wire 2 one by one. When the ring at both ends of the wire are in true circle, the rings tangle with each other, and when the rings are pulled out to the extracting direction, the shoulder part of the ring bump with each other and the it is difficult to pull out the rings. When the rings tangle with each other, the shape deforms, and then it becomes even more difficult to pull out the rings. When the ring shape is made into a teardrop shape, the shoulder part of the ring is slanted and is narrowed, therefore the wires do not tangle with each other and it is easier to pull the rings out of a container case.

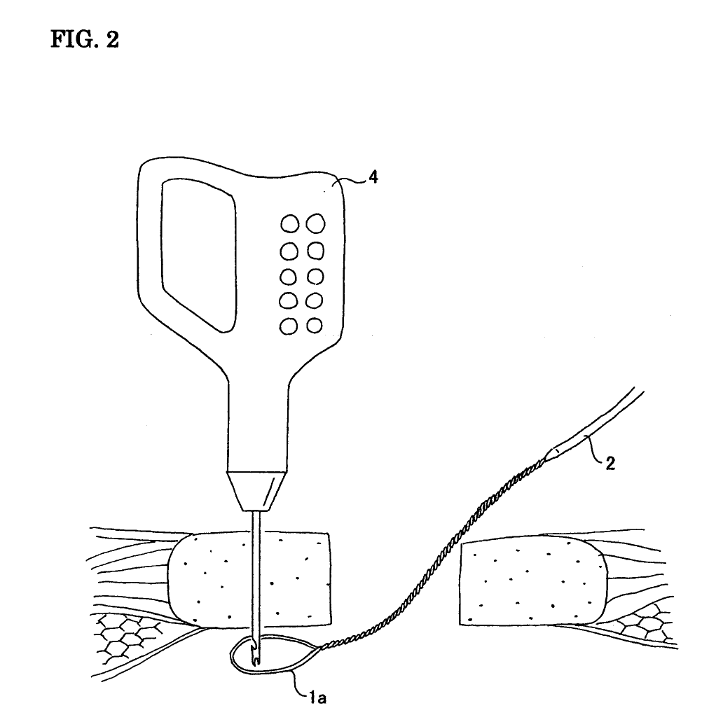

FIG. 2 shows how a wire according to this invention is being used. Gimlet 4 which has a hook at the tip pierces through a sternum, and the wire ring is hooked on at the inside of the sternum, and then the gimlet is pulled out. Same procedure is done at both sides of the incised sternum, and once all the wires are passed through the sternum, the fastening is conducted at the point where the wire is thick.

By shaping the both ends of a sternum suture wire into teardrop shape rings, a multiple number of wires set inside a container case can be taken out without getting tangled. By combining a wire with rings and a gimlet with a hook, the sternum suture in a cardiac surgery can be conducted easily and speedily.

1. A sternum suture wire with teardrop ring, which is a wire used in a sternum closure operation in a cardiac surgery, wherein the center of the wire is thick, the two ends of the wire are thin, and the two tip ends of the wire are shaped in teardrop rings.

2. A manufacturing method of a sternum suture wire with teardrop rings according to claim 1, wherein, when manufacturing the wire, the center of a thin wire is hooked onto the semi-circle part of a pole which has a teardrop cross section, the wire is pulled to the foreside, then the thin wire which has the two ends bundles is fixed to the axis of a motor, the wire is twisted with rotation, and thus teardrop shape rings are formed, the end that is fixed to the motor axis is cut off to a certain specific length, holes are made on both ends of the wire that is thick at the center, the holes being made in a vertical direction with methods such as laser, electrical charge, or drilling, and the outer peripheral of the two cut ends of the twisted teardrop ring wire are attached with pressure onto the two ends of the thick wire.
